(19) United States
(12) Patent Application Publication
(10) Pub. No.: US 2018/0185446 A1
(43) Pub. Date: Jul. 5, 2018

(54) COMPOSITION AND METHOD FOR INDUCING ANTI-APOPTOSIS, SURVIVAL OR PROLIFERATION OF A CELL

(71) Applicant: Vital Therapies, Inc., San Diego, CA (US)

(72) Inventors: Robert A. Ashley, Encinitas, CA (US); Lee Landeen, San Diego, CA (US)

(21) Appl. No.: 15/736,110

(22) PCT Filed: Jun. 14, 2016
(86) PCT No.: PCT/US16/37416

§ 371 Date: Dec. 13, 2017

Related U.S. Application Data
(60) Provisional application No. 62/175,947, filed on Jun. 15, 2015.
(60) Provisional application No. 62/252,219, filed on Nov. 6, 2015.

(51) Int. Cl. A61K 38/18; A61K 38/17; A61P 1/16; A61K 35/407; C12N 5/071

(52) U.S. Cl. CPC A61K 38/1808; A61K 38/177; C12N 5/067; A61K 35/407; A61P 1/16

(57) ABSTRACT

The present invention provides a composition and method for inducing anti-apoptosis, anti-pyroptosis, anti-necroptosis, survival, protection, proliferation, and/or phenotypic modulation of a cell, as well as treating disease in a subject.

### CROSS REFERENCE TO RELATED APPLICATIONS

This application claims the benefit of priority under 35 U.S.C. § 119(e) of U.S. Provisional Patent Application Ser. No. 62/175,947, filed Jun. 15, 2015, and U.S. Provisional Patent Application Ser. No. 62/252,219, filed Nov. 6, 2015, the entire contents of which are incorporated herein by reference in their entireties.

### BACKGROUND

### Field of Invention

The present invention relates generally to cell biology and more particularly to a composition and method for inducing anti-apoptosis, anti-pyroptosis, anti-necroptosis, survival, protection, proliferation, and/or phenotypic modulation of a cell, as well as treating disease in a subject.

### Background Information

Various agents, including but not limited to bacteria, viruses, physical injury, chemical injury (for example, alcohol, drugs and the like), cancer, chemotherapy, and radiation therapy, can, depending on the specific agent and the genetic makeup of the animal exposed to it, cause direct damage to cells and tissue or create an environment of prolonged and excessive inflammation. Under normal conditions, inflammation is a process that helps an animal recover from injury. Acute inflammation is the initial response of a tissue to harmful stimuli. It involves a complex, highly regulated process that begins when cells present in the injured tissue, including macrophages, dendritic cells, histiocytes, Kupffer cells, and mastocytes, sense molecules associated with the injury and become activated. Upon activation, these cells release inflammatory mediators, such as vasodilators. The vasodilators induce increased blood flow and permeability of the blood vessels in the vicinity of the injury. This, in turn, results in the increased movement of plasma and leukocytes (including neutrophils and macrophages) from the blood into the injured tissue. Because inflammatory mediators are, in general, rapidly degraded, acute inflammation requires constant stimulation in order to be sustained. As a result, acute inflammation ends once the harmful stimulus is removed.

Chronic inflammation is believed to be a contributing factor to many widespread and debilitating diseases, including liver diseases, such as hepatitis, cirrhosis and fatty liver disease, heart disease, cancer, respiratory disease, stroke, neurological diseases such as Alzheimer's disease, diabetes, and kidney disease. The result of chronic inflammation is the destruction of normal tissue and its replacement with collagen-rich connective tissue. Collagen-rich connective tissue, also known as scar tissue, exhibits diminished tissue function as compared to normal tissue. Persistent and prolonged formation of scar tissue, in turn, leads to fibrosis. Fibrosis is among the common symptoms of diseases affecting the lungs, skin, liver, heart, and bone marrow, and is a critical factor in diseases such as idiopathic pulmonary fibrosis, scleroderma, keloids, liver cirrhosis, myocardial fibrosis, diabetic kidney disease, myelodysplastic syndrome, and other disorders.

Studies of chronic inflammation and fibrosis have indicated that, regardless of the activating agent and the tissue affected, a common network of signaling proteins tend to function together to establish the pro-inflammatory state. This network of signaling proteins includes a number of different cytokines, cytokine receptors, transcription factors, and the like.

The processing of blood has been performed to remove a variety of blood constituents for therapeutic purposes including inflammatory liver diseases, such as hepatitis. Examples of blood processing methods include hemodialysis that allows to remove metabolic waste products from the blood of patients suffering from inadequate kidney function. Blood flowing from the patient is filtrated to remove these waste products, and then returned to the patient. The method of plasmapheresis also processes blood using tangential flow membrane separation, to treat a wide variety of disease states. Membrane pore sizes can be selected to remove the unwanted plasma constituents. Blood can be also processed using various devices utilizing biochemical reactions to modify biological constituents that are present in blood. For instance, blood components such as bilirubin or phenols can be gluconized or sulfated by the in vitro circulation of blood plasma across enzymes that are bonded to membrane surfaces.

Presently used technologies are generally deficient with respect to supporting patients with compromised liver function, for example. Conventional systems and methods suffer from various problems associated with sustaining such patients until a suitable donor organ can be found for transplantation or until the patient's native liver can regenerate to a healthy state.

The liver possesses enormous capacity to regenerate and replace tissue loss when damaged. Hepatocytes provide the majority of liver functions and respond to regenerative stimuli primarily through cell-surface receptor activation, such as MET (the receptor for hepatocyte growth factor (HGF) and epidermal growth factor receptor (EGFR) which interacts with various ligands). If resident hepatocytes are unable to proliferate, hepatocyte function can be replaced from a regenerative cell pool that is believed to derive from transdifferentated biliary epithelial cells located near the bile ducts. In patients with hepatitis due to chronic alcohol consumption, viral infection, or fulminant toxicants, the resident hepatocytes have a diminished replicative capacity.

Despite growing knowledge about conditions that involve excessive inflammation and/or decreased cell proliferation or survival, treatments for such conditions largely remain elusive. Thus a need exists for more advanced compositions and methods to treat such diseases.

### SUMMARY OF THE INVENTION

In one aspect, the present disclosure provides a composition for inducing anti-apoptosis, anti-pyroptosis, anti-necroptosis, survival, protection, proliferation, and/or phenotypic modulation of a cell. The composition includes one or more anti-apoptotic, anti-pyroptotic, anti-necroptotic, surviving, protective, proliferative, and/or phenotypic modulative factors selected from those set forth in Table I or II.

In another aspect, the present disclosure provides a method of inducing anti-apoptosis, anti-pyroptosis, anti-necroptosis, protection, survival, and/or proliferation, and/or phenotypic modulation of a cell. The method includes contacting the cell with a composition of the disclosure, thereby inducing anti-apoptosis, anti-pyroptosis, anti-necroptosis, protection, survival, and/or proliferation, and/or phenotypic modulation of the cell.

In still another aspect, the present disclosure provides a method of treating a disease or disorder in a subject. The method includes administering a composition of the disclosure to the subject, thereby treating the disease or disorder.

In yet another aspect, the present disclosure provides a qualified C3A cell line derived from a parental C3A cell line, wherein cells of the cell line express one or more factors selected from those set forth in Table I or II in response to pro-inflammatory molecules, such as pro-inflammatory cytokines.

### BRIEF DESCRIPTION OF THE DRAWINGS

FIG. 1 is a graphical plot depicting data relating to an embodiment of the invention.

FIG. 2 is a graphical plot depicting data relating to an embodiment of the invention.

FIG. 3 is a simplified block diagram illustrating a prior art extracorporeal filtration and detoxification system.

### DETAILED DESCRIPTION OF THE INVENTION

The present invention is based on the unexpected finding that cells of a certain C3A clonal cell line are capable of producing a variety of secreted factors with involvement in liver regeneration and hepatocyte proliferation. These factors facilitate liver regeneration, either directly through direct stimulation of hepatocytes or indirectly through interactions with other resident cell populations upon introduction into the circulatory system of a subject. This knowledge forms the basis for providing a composition and method for inducing anti-apoptosis, anti-pyroptosis, anti-necroptosis, protection, survival, and/or proliferation, and/or phenotypic modulation of a cell, as well as treatment of disease.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

The principles and operation of the methods according to the present disclosure may be better understood with reference to the figures and accompanying descriptions.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of skill in the art to which this disclosure belongs. Although any methods and materials similar or equivalent to those described herein can be used in the practice or testing of the disclosure, some preferred methods and materials are now described.

The invention described herein relates to a composition which includes one or more anti-apoptotic, pro-survival, and/or pro-regeneration factors. The composition may be used to produce pharmaceutical compositions for use in treating a disease, disorder, or otherwise abnormal condition, such as an inflammatory disease or disorder.

As used herein, the term “subject” refers to a mammalian subject. As such, treatment of any animal in the order mammalian is envisioned. Such animals include, but are not limited to horses, cats, dogs, rabbits, mice, goats, sheep, non-human primates and humans. Thus, the method of the present disclosure is contemplated for use in veterinary applications as well as human use.

“Treatment” of a subject herein refers to both therapeutic treatment and prophylactic or preventative measures. Those in need of treatment include those already with a disease or disorder as well as those in which it is to be prevented. Hence, the subject may have been diagnosed as having a disease or disorder or may be predisposed or susceptible to a disease or disorder.

The expression “effective amount” refers to an amount of an anti-apoptotic, pro-survival, and/or pro-regeneration factor, that is effective for preventing, ameliorating or treating a disease or disorder. Such an effective amount will generally result in an improvement in the signs, symptoms or other indicators of a disease or disorder. For example, in liver diseases, an effective amount results in the reduction of biochemical markers indicative or poor hepatic function.

A “symptom” of a disease or disorder is any morbid phenomenon or departure from the normal in structure, function, or sensation, experienced by the subject and indicative of a disease or disorder.

As used herein, “inflammatory disease, disorder, or otherwise abnormal condition,” may include disorders associated with inflammation or have an inflammation component, such as, but are not limited to: sepsis, infection (such as viral, bacterial or fungal infection), acne vulgaris, asthma, chronic obstructive pulmonary disease (COPD), autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, and ischaemic heart disease, type I diabetes, and diabetic nephropathy.

In certain embodiments, the inflammatory disease, disorder, or otherwise abnormal condition includes many autoimmune diseases or disorders that are associated with inflammation or have an inflammation component, e.g., corresponding to one or more types of hypersensitivity. Exemplary autoimmune diseases or disorders that correspond to one or more types of hypersensitivity include: atopic allergy, atopic dermatitis, autoimmune hemolytic anemia, autoimmune hepatitis, autoimmune polyendocrine syndrome, autoimmune urticaria, celiac disease, cold agglutinin disease, contact dermatitis, Crohn's disease, diabetes mellitus type 1, discoid lupus erythematosus, Erythroblastosis fetalis, Goodpasture's syndrome, Graves' disease, Guillain-Barre syndrome (GBS), Hashimoto's encephalopathy, Hashimoto's thyroiditis, idiopathic thrombocytopenic purpura, autoimmune thrombocytopenic purpura, IgA nephropathy, lupus erythematosus, Meniere's disease, multiple sclerosis, myasthenia gravis, narcolepsy, neuromyelitis optica, Devic's disease, neuromyotonia, ocular cicatricial pemphigoid, opsoclonus myoclonus syndrome, PANDAS (pediatric autoimmune neuropsychiatric disorders associated with streptococcus), paraneoplastic cerebellar degeneration, pemphigus vulgaris, pernicious anaemia, psoriasis, psoriatic arthritis, rheumatoid arthritis, rheumatic fever, sarcoidosis, scleroderma, subacute bacterial endocarditis (SBE), systemic lupus erythematosis, Lupus erythematosis, temporal arteritis (also known as “giant cell arteritis”), thrombocytopenia, ulcerative colitis, undifferentiated connective tissue disease, urticarial vasculitis, and vasculitis.

Inflammatory disease, disorder, or otherwise abnormal condition in liver may include fatty liver disease, cirrhosis, liver cancer, and acute or chronic hepatitis caused by viral infection (e.g., by Hepatitis A, B, C, D and E), alcoholic hepatitis, drug or chemical intoxication (such as carbon-tetrachloride, amethopterin, tetracycline, acetaminophen, fenoprofen, and the like), mononucleosis, amebic dysentery, and other systematic infections by Epstein-Barr virus (EBV), cytomegalovirus (CMV), or bacteria.

Inflammatory disease, disorder, or otherwise abnormal condition in kidney may be associated with acute or chronic nephritis, interstitial nephritis, lupus nephritis, IgA nephropathy (Berger's disease), glomerulonephritis, membranoproliferative glomerulonephritis (MPGN), autoimmune disorders related to chronic kidney disease (CKD) and inflammation, Goodpasture's syndrome, Wegener's granulomatosis, pyelonephritis, athletic nephritis, kidney stones, and gout.

Inflammatory bowel disease (IBD) is a group of inflammatory conditions of the colon and small intestine. The major types of IBD are Crohn's disease and ulcerative colitis. Other forms of IBD, which are not always classified as typical IBD, include collagenous colitis, lymphocytic colitis, ischaemic colitis, diversion colitis, Behcet's disease, and indeterminate colitis.

Inflammatory disease, disorder, or otherwise abnormal condition in pancreas includes various forms of pancreatitis with a variety of causes and symptoms, including pancreatitis caused by alcohol, gallstone, medication (e.g., use of corticosteroids such as prednisolone, HIV drugs such as didanosine and pentamidine, diuretics, the anticonvulsant valproic acid, the chemotherapeutic agents L-asparaginase and azathioprine, estrogen by way of increased blood triglycerides, cholesterol-lowering statins, and the antihyperglycemic agents like metformin, vildagliptin, sitagliptin, and diabetes drug gliptins), trauma, mumps, autoimmune disease, scorpion stings, high blood calcium, high blood triglycerides, hypothermia, endoscopic retrograde cholangiopancreatography (ERCP), Pancreas divisum, pregnancy, diabetes mellitus type 2, pancreatic cancer, pancreatic duct stones, vasculitis (inflammation of the small blood vessels in the pancreas), coxsackievirus infection, and porphyria—particularly acute intermittent porphyria and erythropoietic protoporphyria, viral infection (by coxsackie virus, cytomegalovirus, Hepatitis B, herpes simplex virus, mumps, varicella-zoster virus), bacterial infection (Legionella, Leptospira, Mycoplasma, Salmonella), fungal infection (Aspergillus), or parasitic infection (Ascaris, Cryptosporidium, Toxoplasma).

The present invention provides a composition which includes one or more anti-apoptotic, pro-survival, and/or pro-regeneration factors, which are generally peptides. In embodiments, the one or more factors are selected from those set forth in Table I or II, any may include any combination thereof

TABLE I
Factors of the disclosure (e.g., anti-apoptotic,
pro-survival, and/or pro-regeneration factors).
Factors

Amphiregulin (AR)
Soluble Fas (sFAS) Receptor
Albumin
Alpha-1-Antitrypsin (AAT)
Alpha-2-Macroglobulin (A2Macro)
Alpha-Fetoprotein (AFP)
Angiopoeitin-2 (ANG-2)
Apolipoprotein A-I (Apo A-I)
Apolipoprotein A-II (Apo A-II)
Apolipoprotein C-I (Apo C-I)
Apolipoprotein C-III (Apo C-III)
Apolipoprotein H (Apo H)
Beta-2-Microglobulin (β2M)
CD 40 antigen (CD40)
Complement C3 (C3)
CreatineKinase-MB (CK-MB)
Eotaxin-1
Erythropoietin (EPO)
Factor VII
Ferritin (FRTN)
Fibrinogen
Gelsolin
Hepatocyte Growth Factor (HGF)
Heparin Binding Epidermal Growth Factor
(HB-EGF)
Human Chorionic Gonadotropin beta (hCG)
Intercellular Adhesion Molecule 1 (ICAM-1)
Interleukin-1 receptor antagonist (IL-1Ra)
Interleukin-8 (IL-8)
Macrophage-Derived Chemokine (MDC)
Neuron-Specific Enolase (NSE)
Neutrophil Gelatinase-associated Lipocalin (NGAL)
Placental Growth Factor (PLGF)
Plasminogen Activator Inhibitor 1 (PAI-1)
Platelet-derived Growth Factor BB (PDGF-BB)
Serotransferrin (Transferrin)
Sex Hormone-Binding Globulin (SHBG)
Stem Cell Factor (SCF)
T-Cell-Specific Protein RANTES (RANTES)
Thyroxine-Binding Globulin (TBG)
Tissue Inhibitor of Metalloproteinse 1 (TIMP-1)
Transforming Growth Factor alpha (TGFα)
Transthyretin (TTR)
Vascular Endothelial Growth Factor (VEGF)
Vascular Endothelial Growth Factor C (VEGF-C)

TABLE II
Factors of the disclosure (e.g., anti-apoptotic,
pro-survival, and/or pro-regeneration factors).
Factors

Amphiregulin (AR)
Soluble Fas (sFAS) Receptor
Alpha-1-Antitrypsin (AAT)
Angiopoeitin-2 (ANG-2)
Erythropoietin (EPO)
Gelsolin
Hepatocyte Growth Factor (HGF)
Heparin Binding Epidermal Growth Factor (HB-EGF)
Interleukin-1 receptor antagonist (IL-1Ra)
Placental Growth Factor (PLGF)
Platelet-derived Growth Factor BB (PDGF-BB)
Stem Cell Factor (SCF)
Transforming Growth Factor alpha (TGFα)
Vascular Endothelial Growth Factor (VEGF)
Vascular Endothelial Growth Factor C (VEGF-C)

In various embodiments the one or more factors includes at least AR or sFAS. In one embodiment the one or more factors includes both AR and sFAS and optionally one or more additional factors from Table I or II, such as a known mitogen, a factor that inhibits apoptosis related signal transduction in non-disease related cells, a factor that promotes apoptosis in disease related cells, and/or a factor that induces a phenotypic shift resulting in improved cellular functioning.

In one embodiment, the one or more factors includes all of those set forth in Table I. In one embodiment, the one or more factors includes all of those set forth in Table II.

In one embodiment, the one or more factors includes AR, sFAS and one or more of Hepatocyte growth factor (HGF), Transforming growth factor alpha, Heparin binding epidermal growth factor, Platelet-derived growth factor BB, Vascular endothelial growth factor, Vascular endothelial growth factor C, Placental growth factor, Angiopoietin2, Erythropoietin, Stem cell factor or any combination thereof. In embodiments, the one or more factors includes AR, sFAS, Hepatocyte growth factor (HGF), Transforming growth factor alpha, Heparin binding epidermal growth factor, Platelet-derived growth factor BB, Vascular endothelial growth factor, Vascular endothelial growth factor C, Placental growth factor, Angiopoietin2, Erythropoietin and Stem cell factor.

In one embodiment the one or more factors includes AR, sFAS and one or more of AAT, A2Macro, Apo A-I, Apo A-II, Apo C-I, Apo C-III, Apo H, β2M, Cancer Antigen 125 (CA-125), CD 40 antigen (CD40), CreatineKinase-MB (CK-MB), Eotaxin-1, Factor VII, Ferritin (FRTN), Fibrinogen, ICAM-1, IL-1Ra, IL-7, IL-8, IL-17, Macrophage-Derived Chemokine (MDC), Neuron-Specific Enolase (NSE), Plasminogen Activator Inhibitor 1 (PAI-1), Serotransferrin (Transferrin), Sex Hormone-Binding Globulin (SHBG), Thyroxine-Binding Globulin (TBG), TIMP-1, Transthyretin (TTR), or any combination thereof. In one embodiment the one or more factors includes AR, sFAS, AAT, A2Macro, Apo A-I, Apo A-II, Apo C-I, Apo C-III, Apo H, β2M, Cancer Antigen 125 (CA-125), CD 40 antigen (CD40), CreatineKinase-MB (CK-MB), Eotaxin-1, Factor VII, Ferritin (FRTN), Fibrinogen, ICAM-1, IL-1Ra, IL-7, IL-8, IL-17, Macrophage-Derived Chemokine (MDC), Neuron-Specific Enolase (NSE), Plasminogen Activator Inhibitor 1 (PAI-1), Serotransferrin (Transferrin), Sex Hormone-Binding Globulin (SHBG), Thyroxine-Binding Globulin (TBG), TIMP-1 and Transthyretin (TTR).

In embodiments the one or more factors are polypeptides, such as those set forth in Table 1. In embodiments, the composition is a pharmaceutical composition that includes one or more factors, such as a polypeptide and a pharmaceutically acceptable carrier. The terms “polypeptide”, “peptide”, or “protein” are used interchangeably herein to designate a linear series of amino acid residues connected one to the other by peptide bonds between the alpha-amino and carboxy groups of adjacent residues.

In embodiments, the composition includes a single type of factor from Table I, such as AR or sFAS. In other embodiments, the pharmaceutical composition includes a combination of two or more factors from Table I, such as AR and sFas. In embodiments, the composition is substantially free of blood proteins and/or metabolites found in the blood. In other embodiments, the composition includes serum albumin (e.g., human serum albumin). In embodiments, any polypeptide factor present in the composition is recombinantly produced. In embodiments, any polypeptide factor present in the composition is produced by a C3A cell in response to blood, or fraction thereof, from a subject.

The composition may further include one or more agents that increase expression or activity of one or more of the factors set forth in Table I. An agent useful in the invention can be any type of molecule, for example, a polynucleotide, a peptide, a peptidomimetic, peptoids such as vinylogous peptoids, chemical compounds, such as organic molecules or small organic molecules, or the like. In various embodiments, expression or activity is increased by a factor of at least 2.0, 5.0, 10, 25, 50, 100, 250, 500, 1,000, 5,000 or greater as compared to expression or activity prior to contacting with the agent.

In embodiments, the agent is a polynucleotide, such as an antisense oligonucleotide or RNA molecule which increases expression and/or activity (directly or indirectly) in a cell of a factor set forth in Table I. In various aspects, the agent may be a polynucleotide, such as an antisense oligonucleotide or RNA molecule, such as microRNA, dsRNA, siRNA, stRNA, and shRNA.

MicroRNAs (miRNA) are single-stranded RNA molecules, which regulate gene expression. miRNAs are encoded by genes from whose DNA they are transcribed but miRNAs are not translated into protein; instead each primary transcript (a pri-miRNA) is processed into a short stem-loop structure called a pre-miRNA and finally into a functional miRNA. Mature miRNA molecules are either fully or partially complementary to one or more messenger RNA (mRNA) molecules, and their main function is to down-regulate gene expression. MicroRNAs can be encoded by independent genes, but also be processed (via the enzyme Dicer) from a variety of different RNA species, including introns, 3′ UTRs of mRNAs, long noncoding RNAs, snoRNAs and transposons. As used herein, microRNAs also include “mimic” microRNAs which are intended to mean a microRNA exogenously introduced into a cell that have the same or substantially the same function as their endogenous counterpart. Thus, while one of skill in the art would understand that an agent may be an exogenously introduced RNA, an agent also includes a compound or the like that increase or decrease expression of microRNA in the cell.

The terms “small interfering RNA” and “siRNA” also are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome).

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

As discussed herein, the composition of the disclosure can include a single factor set forth in Table I, or combinations thereof. The composition can be substantially free of proteins other than those of Table I. The composition can be substantially free of any pro-inflammatory molecules. As used herein, the term “substantially free of proteins other than those of Table I” means that less than 5% of the protein content of the composition is made up of proteins that are not set forth in Table I. As used herein, the term “substantially free of a pro-inflammatory molecule” means that less than 5% of the content of the composition is made up of pro-inflammatory molecules. A composition that is substantially free of proteins other than those of Table I can have less than 4%, 3%, 2%, 1%, 0.5%, 0.1%, 0.05%, 0.01%, 0.001%, 0.0001%, or less (e.g., 0.0%) of proteins other than those of Table I. A composition that is substantially free of a pro-inflammatory molecule can have less than 4%, 3%, 2%, 1%, 0.5%, 0.1%, 0.05%, 0.01%, 0.001%, 0.0001% or less of such molecules. Thus, the composition can be substantially free of blood proteins, such as serum albumin, globulins, fibrinogen, and clotting factors. Alternatively, the composition can include one or more of serum albumin, globulins, fibrinogen, and clotting factors.

In embodiments, the peptide factor of the composition is not naturally found in a human or other mammal or animal. For example, the factor may be synthetic, recombinant or the like. However, a composition of the invention can include a peptide factor that is naturally found in a human or other mammal or animal.

In embodiments, the peptide factor may include a non-naturally occurring amino acid. “Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

In embodiments, the composition includes one or more conservatively modified variants of a factor set forth in Table I. In embodiments, the conservatively modified variant has at least 80% sequence similarity, often at least 85% sequence similarity, 90% sequence similarity, or at least 95%, 96%, 97%, 98%, or 99% sequence similarity at the amino acid level, with the naturally occurring polypeptide.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or percent “identity,” in the context of two or more polypeptide sequences, refer to two or more sequences or subsequences that are the same or have a specified percentage of amino acid residues that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithm with default parameters, or by manual alignment and visual inspection. Such sequences are then said to be “substantially identical.”

In embodiments, the composition is substantially free of biological molecules (such as polypeptides, nucleic acids, lipids, carbohydrates, and metabolites) that are associated with the one or more factors of the invention in vivo or co-purify with the factors. As used herein, the term “substantially free of biological molecules” means that less than 5% of the dry weight of the composition is made up of biological molecules not set forth in Table I. A composition that is substantially free of such biological molecules can have less than 4%, 3%, 2%, 1%, 0.5%, 0.1%, 0.05%, 0.01%, or less of biological molecules that are not set forth in Table I. Thus, for example, the composition can be substantially free of biological molecules that are abundant in the blood, such as, fatty acids, cholesterol, non-protein clotting factors, metabolites, and the like. In addition, the composition can be substantially free of cells, including red blood cells, white blood cells, platelets, and cell fragments.

In embodiments, the composition of the invention includes at least 1 mg (e.g., at least 5, 10, 20, 30, 40, 50, 75, 100, 150, 200, 250, 300, 400, 500, 600, 700, 800, 900, 1000 mg, or more) of one or more of the factors set forth in Table I. Thus, for example, the composition can include an amount of one or more factors equal to about 1 mg to about 1000 mg (e.g., about 5 mg to about 900 mg, about 5 mg to about 800 mg, about 5 mg to about 700 mg, about 5 mg to about 600 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 75 mg to about 500 mg, about 75 mg to about 400 mg, about 75 mg to about 300 mg, about 75 mg to about 250 mg, about 75 mg to about 200 mg, about 75 mg to about 150 mg, about 75 mg to about 100 mg, about 100 mg to about 500 mg, about 100 mg to about 400 mg, about 100 mg to about 300 mg, about 100 mg to about 250 mg, about 100 mg to about 200 mg, or any other range containing two of the foregoing endpoints).

In embodiments, the composition of the invention can include a solution that contains at least 1 mg/ml (e.g., at least 5, 10, 15, 20, 25, 30, 35, 40, 45, 50, 55, 60, 65, 70, 75, 80, 85, 90, 95, 100 mg/ml or more) of one or more of the factors set forth in Table I. Thus, for example, the composition can include a solution having a concentration of one or more of the factors set forth in Table I of about 1 mg/ml to about 1000 mg/ml (e.g., about 5 mg/ml to about 900 mg/ml, about 5 mg/ml to about 800 mg/ml, about 5 mg/ml to about 700 mg/ml, about 5 mg/ml to about 600 mg/ml, about 5 mg/ml to about 500 mg/ml, about 10 mg/ml to about 500 mg/ml, about 10 mg/ml to about 400 mg/ml, about 10 mg/ml to about 300 mg/ml, about 10 mg/ml to about 250 mg/ml, about 10 mg/ml to about 200 mg/ml, about 10 mg/ml to about 150 mg/ml, about 10 mg/ml to about 100 mg/ml, about 50 mg/ml to about 500 mg/ml, about 50 mg/ml to about 400 mg/ml, about 50 mg/ml to about 300 mg/ml, about 50 mg/ml to about 250 mg/ml, about 50 mg/ml to about 200 mg/ml, about 50 mg/ml to about 150 mg/ml, about 50 mg/ml to about 100 mg/ml, about 75 mg/ml to about 500 mg/ml, about 75 mg/ml to about 400 mg/ml, about 75 mg/ml to about 300 mg/ml, about 75 mg/ml to about 250 mg/ml, about 75 mg/ml to about 200 mg/ml, about 75 mg/ml to about 150 mg/ml, about 75 mg/ml to about 100 mg/ml, about 100 mg/ml to about 500 mg/ml, about 100 mg/ml to about 400 mg/ml, about 100 mg/ml to about 300 mg/ml, about 100 mg/ml to about 250 mg/ml, about 100 mg/ml to about 200 mg/ml, about 10 mg/ml to about 150 mg/ml, or any other range containing two of the foregoing endpoints).

In embodiments, the composition of the invention includes at least 1 pg (e.g., at least 5, 10, 20, 30, 40, 50, 75, 100, 150, 200, 250, 300, 400, 500, 600, 700, 800, 900, 1000 pg, or more) of one or more of the factors set forth in Table I. Thus, for example, the composition can include an amount of one or more factors of equal to about 1 pg to about 1000 pg (e.g., about 5 pg to about 900 pg, about 5 pg to about 800 pg, about 5 pg to about 700 pg, about 5 pg to about 600 pg, about 10 pg to about 500 pg, about 10 pg to about 400 pg, about 10 pg to about 300 pg, about 10 pg to about 250 pg, about 10 pg to about 200 pg, about 10 pg to about 150 pg, about 10 pg to about 100 pg, about 50 pg to about 500 pg, about 50 pg to about 400 pg, about 50 pg to about 300 pg, about 50 pg to about 250 pg, about 50 pg to about 200 pg, about 50 pg to about 150 pg, about 50 pg to about 100 pg, about 75 pg to about 500 pg, about 75 pg to about 400 pg, about 75 pg to about 300 pg, about 75 pg to about 250 pg, about 75 pg to about 200 pg, about 75 pg to about 150 pg, about 75 pg to about 100 pg, about 100 pg to about 500 pg, about 100 pg to about 400 pg, about 100 pg to about 300 pg, about 100 pg to about 250 pg, about 100 pg to about 200 pg, or any other range containing two of the foregoing endpoints).

In embodiments, the composition of the invention can include a solution that contains at least 1 pg/ml (e.g., at least 5, 10, 15, 20, 25, 30, 35, 40, 45, 50, 55, 60, 65, 70, 75, 80, 85, 90, 95, 100 pg/ml or more) of one or more of the factors set forth in Table I. Thus, for example, the composition can include a solution having a concentration of one or more of the factors set forth in Table I of about 1 pg/ml to about 1000 pg/ml (e.g., about 5 pg/ml to about 900 pg/ml, about 5 pg/ml to about 800 pg/ml, about 5 pg/ml to about 700 pg/ml, about 5 pg/ml to about 600 pg/ml, about 5 pg/ml to about 500 pg/ml, about 10 pg/ml to about 500 pg/ml, about 10 pg/ml to about 400 pg/ml, about 10 pg/ml to about 300 pg/ml, about 10 pg/ml to about 250 pg/ml, about 10 pg/ml to about 200 pg/ml, about 10 pg/ml to about 150 pg/ml, about 10 pg/ml to about 100 pg/ml, about 50 pg/ml to about 500 pg/ml, about 50 pg/ml to about 400 pg/ml, about 50 pg/ml to about 300 pg/ml, about 50 pg/ml to about 250 pg/ml, about 50 pg/ml to about 200 pg/ml, about 50 pg/ml to about 150 pg/ml, about 50 pg/ml to about 100 pg/ml, about 75 pg/ml to about 500 pg/ml, about 75 pg/ml to about 400 pg/ml, about 75 pg/ml to about 300 pg/ml, about 75 pg/ml to about 250 pg/ml, about 75 pg/ml to about 200 pg/ml, about 75 pg/ml to about 150 pg/ml, about 75 pg/ml to about 100 pg/ml, about 100 pg/ml to about 500 pg/ml, about 100 pg/ml to about 400 pg/ml, about 100 pg/ml to about 300 pg/ml, about 100 pg/ml to about 250 pg/ml, about 100 pg/ml to about 200 pg/ml, about 10 pg/ml to about 150 pg/ml, or any other range containing two of the foregoing endpoints).

The composition of the invention is typically a pharmaceutical composition. Such a pharmaceutical composition can include one or more of the factors set forth in Table I or II and a pharmaceutically acceptable carrier. A pharmaceutical composition can further include a protein other than a factor as set forth in Table I or II. The other protein can be a therapeutic agent, such as a therapeutic polypeptide. Alternatively, the other protein can be a carrier protein.

In embodiments, the composition of the invention includes an anti-coagulant, such as heparin or citrate. As used herein, “citrate” refers to a citrate anion, in any form, including citric acid (citrate anion complexed with three protons), salts containing citrate anion, and partial casters of citrate anion. Citrate anion is an organic tricarboxylate. Citric acid, which has been assigned Chemical Abstracts Registry No. 77-92-2, has the molecular formula HOC(CO₂H)(CH₂CO₂H)₂ and a formula weight of 192.12 g/mol. A citrate salt (i.e., a salt containing citrate anion) is composed of one or more citrate anions in association with one or more physiologically-acceptable cations. Exemplary physiologically-acceptable cations include, but are not limited to, protons, ammonium cations and metal cations. Suitable metal cations include, but are not limited to, sodium, potassium, calcium, and magnesium, where sodium and potassium are preferred, and sodium is more preferred. A composition containing citrate anion may contain a mixture of physiologically-acceptable cations.

In one embodiment, the composition includes sodium citrate. Sodium citrate may be in the form of a dry chemical powder, crystal, pellet or tablet. Any physiologically tolerable form of citric acid or sodium citrate may be used. For instance, the citric acid or sodium citrate may be in the form of a hydrate, including a monohydrate.

The pharmaceutical composition of the invention may be prepared by mixing one or more of the factors set forth in Table I having the desired degree of purity with optional pharmaceutically acceptable carriers, excipients or stabilizers (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

In embodiments, the composition of the present invention may include live cells. In one embodiment, the composition includes a hepatocyte cell. In one embodiment, the composition includes HepG2 cells or C3A cells which are optionally recombinantly engineered.

The composition of the invention provides powerful tools for inducing anti-apoptosis, survival, and/or proliferation of a target cell and/or treating a disease or disorder, such as an inflammatory disease.

Accordingly, the invention provides a method of inducing anti-apoptosis, anti-pyroptosis, anti-necroptosis, survival, protection, proliferation, and/or phenotypic modulation in a cell by contacting the cell with a composition of the disclosure. In embodiments, proliferation of the contacted cell is increase by a factor of at least 1.1, 1.5, 2.0, 5.0, 10, 25, 50, 100 or greater as compared to proliferation of a comparable cell not contacted with the composition. In a related embodiment, survival of the cell is increase by a factor of at least 1.1, 1.5, 2.0, 5.0, 10, 25, 50, 100 or greater as compared to survival of a comparable cell not contacted with the composition.

The invention also provides a method of treating a disease or disorder in a subject. The method includes administering one or more factors set forth in Table I or II (or, for example, a pharmaceutical composition comprising one or more factors set forth in Table I or II) to the subject, or cell or tissue thereof.

In the method of the invention, the one or more factors induces anti-apoptosis, anti-pyroptosis, anti-necroptosis, survival, protection, proliferation, and/or phenotypic modulation in the contacted cell or tissue. In embodiments, the contacted cell (also referred to as a target cell) is a eukaryotic cell, such as a mammalian cell. In one embodiment the contacted cell is a hepatocyte. In one embodiment, the cell is a hepatoblastoma-derived cell. In one embodiment, the cell is a HepG2 cell or a C3A cell of a C3A cell line. In one embodiment, the cell is a clonal derivative from a parental C3A cell line. In one embodiment, the cell is a recombinantly engineered cell.

The term “C3A cell line” refers to a sub-clone of the human hepatoblastoma cell line HepG2. The C3A cell line is a qualified cell line having been deposited at the American Type Culture Collection under ATCC No. CRL-10741.

Administration of the composition may be performed in any suitable manner including, for example, intravenously, intraperitoneally, parenteral, orthotopically, subcutaneously, topically, nasally, orally, sublingually, intraocularly, by means of an implantable depot, using nanoparticle-based delivery systems, microneedle patch, microspheres, beads, osmotic or mechanical pumps, and/or other mechanical means.

In various embodiments, a cell may be contacted by the composition in-vivo or in-vitro. In one embodiment, the cell is contacted in-vivo, the contacted cell being within a subject being treated by an extracorporeal detoxification system, such as that described in U.S. Pat. No. 8,105,491 which is incorporated herein by reference in its entirety. In such embodiments, one more factors of the composition may be produced by a cell, such as a C3A cell, contained within an active cartridge (bioreactor) of the system. In various embodiments, the system may be fluidly coupled to a subject, or a cell or organ thereof, e.g., a liver.

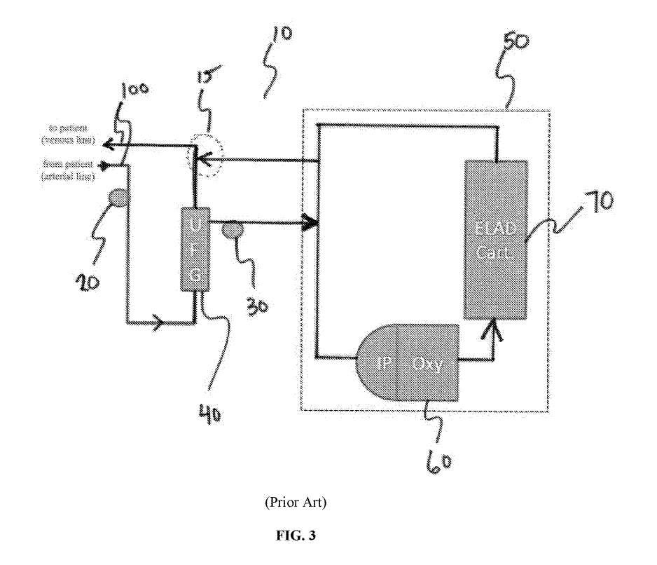

As indicated in FIG. 3, the extracorporeal detoxification system 10 generally includes a blood circuit 100 configured to be coupled to a patient and operative to communicate blood from the patient, through an ultrafiltrate generator (UFG) 40, and back to the patient; a recirculation circuit 50 coupled to the UFG 40 and operative to draw ultrafiltrate from the UFG 40 and to treat ultrafiltrate independently of cellular components of the blood; and a conduit junction 15 operative to recombine the ultrafiltrate in the recirculation circuit 50 and the cellular components in the blood circuit 100 prior to reintroduction to the patient. Also shown in FIG. 3 is an active cartridge 70 and oxygenator 60 arranged within the recirculation circuit 50. The active cartridge 70 is utilized to treat the ultrafiltrate.

The term “active cartridge” refers to a hollow fiber based cartridge comprising cells (such as, for example, cells of the C3A cell line) having utility in therapeutic applications and detoxification processes.

The term “blood circuit” refers to a circuit of tubing connected to a double lumen catheter and operative to circulate blood from a patient to a blood control unit and back to the patient.

The term “C3A cell line” refers to a sub-clone of the human hepatoblastoma cell line HepG2. In embodiments, C3A cells are contained in the extracapillary space of one or more active cartridges. The C3A cell line has been deposited at the American Type Culture Collection under ATCC No. CRL-10741.

The term “detoxification device” refers to a cartridge, canister, or other device that provides a means of removal of specific or non-specific molecules from a fluid stream. Examples would be a dialysis cartridge, an adsorption cartridge, or a filter.

The term “extracapillary space” (ECS) refers to space outside the hollow fibers of active cartridges or an ultrafiltrate generator. The ECS of active cartridges may generally house the C3A cells.

The term “intracapillary space” (ICS) refers to space inside the hollow fibers of active cartridges or an ultrafiltrate generator. The ICS is the flow path for whole blood or the ultrafiltrate fluid.

The term “recirculation circuit” refers to a circuit generally enabling filtration, detoxification, and treatment of ultrafiltrate fluid; in some implementations, a recirculation circuit generally encompasses a reservoir, an oxygenator, and one or more active cartridges.

The term “ultrafiltrate” (UF) refers to plasma fluid and dissolved macromolecules filtered across the semi-permeable membrane of an ultrafiltrate generator.

The term “ultrafiltrate generator” (UFG) refers to a device comprising or embodied as a “blank” active cartridge (i.e., a hollow fiber cartridge which does not contain therapeutically active cells) and operative to separate plasma fluid (ultrafiltrate) from cellular blood components. The hollow fibers may be composed of a semi-permeable membrane which has, for example, a nominal molecular weight cut-off of approximately 100,000 Daltons in some implementations. During use of the UFG, blood may be circulated through the ICS of the hollow fibers; ultrafiltrate, comprising blood plasma and various macromolecules, passes through the membrane fiber walls into the recirculation circuit, where it is circulated through one or more active cartridges.

The term “ultrafiltration” refers generally to a process during which ultrafiltrate is pulled from whole blood across the semi-permeable membrane of the UFG. In some embodiments described below, an ultrafiltrate pump may control the rate of ultrafiltrate production, while the pore size of the hollow fiber membrane of the UFG may control the amount of ultrafiltrate permeating the membrane.

During clinical or therapeutic treatment, UF may be pumped through the lumen (ICS) of the hollow fiber cartridge within the active cartridge 70, allowing toxins, nutrients, glucose, and dissolved oxygen from the UF to diffuse across the membrane into the ECS, where the live cells may metabolize them. Metabolites, along with albumin and other proteins produced by the cells, may diffuse back across the membrane into the UF for return to the patient.

As set forth above and contemplated herein, the C3A cell line is a subclone of the human hepatoblastoma cell line HepG2. Some subclones of this parent cell line, such as C3A, for example, exhibit liver-specific functional capabilities such as high albumin production and α-fetoprotein (AFP) production as well as expression of anti-inflammatory mediator proteins α-1-antitrypsin (AAT) and IL-1Ra in response to pro-inflammatory molecules of the present invention, including for example, cytokines IL-6 and IL-1β. Such cells are also capable of producing one or more factors set forth in Table I or II.

In various embodiments, the system may be fluidly coupled to the subject, or a cell or organ thereof, e.g., a liver. The composition of the present invention is introduced into the blood circuit of system 10. The composition may be introduced into the circulatory of the subject, or introduced directly into the blood flow path of the system. In one embodiment, one or more of the factors set forth in Table I or II is generated by cells within the active cartridge 70 of system 10. Once in the blood circuit 100 of system 10, treated UF including factors of the composition is reintroduced into the subject wherein the factors of the composition contact cells of the subject, such as liver cells, thereby facilitating treatment of a disease or disorder.

While the cells of the active cartridge are illustrated as being C3A cells in the present embodiment, one of skill in the art would understand that the active cartridge could include any number of suitable cell types which are beneficial in treating any number of different diseases, such as inflammatory diseases as disclosed herein. In embodiments, the active cartridge may include cells recombinantly engineered to produce one or more of the factors set forth in Table I or II, such as AR and/or sFas, in response to a stimuli, for example, a stimuli generated within the subject being treated, such as a pro-inflammatory molecule.

In conjunction with any of the foregoing methods, the composition can be administered daily (or every other day, or weekly), wherein the amount of one or more factors of Table I or II is between about 1 mg and about 1000 mg (e.g., about 5 mg to about 900 mg, about 5 mg to about 800 mg, about 5 mg to about 700 mg, about 5 mg to about 600 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 75 mg to about 500 mg, about 75 mg to about 400 mg, about 75 mg to about 300 mg, about 75 mg to about 250 mg, about 75 mg to about 200 mg, about 75 mg to about 150 mg, about 75 mg to about 100 mg, about 100 mg to about 500 mg, about 100 mg to about 400 mg, about 100 mg to about 300 mg, about 100 mg to about 250 mg, about 100 mg to about 200 mg, or any other range containing two of the foregoing endpoints).

In conjunction with any of the foregoing methods, the composition can be administered daily (or every other day, or weekly), wherein the amount of one or more factors of Table I or II is between about 1 pg and about 1000 pg (e.g., about 5 pg to about 900 pg, about 5 pg to about 800 pg, about 5 pg to about 700 pg, about 5 pg to about 600 pg, about 10 pg to about 500 pg, about 10 pg to about 400 pg, about 10 pg to about 300 pg, about 10 pg to about 250 pg, about 10 pg to about 200 pg, about 10 pg to about 150 pg, about 10 pg to about 100 pg, about 50 pg to about 500 pg, about 50 pg to about 400 pg, about 50 pg to about 300 pg, about 50 pg to about 250 pg, about 50 pg to about 200 pg, about 50 pg to about 150 pg, about 50 pg to about 100 pg, about 75 pg to about 500 pg, about 75 pg to about 400 pg, about 75 pg to about 300 pg, about 75 pg to about 250 pg, about 75 pg to about 200 pg, about 75 pg to about 150 pg, about 75 pg to about 100 pg, about 100 pg to about 500 pg, about 100 pg to about 400 pg, about 100 pg to about 300 pg, about 100 pg to about 250 pg, about 100 pg to about 200 pg, or any other range containing two of the foregoing endpoints).

In conjunction with any of the foregoing methods, the composition can be administered in combination with a drug useful for treatment of the disease or disorder. In one embodiment, the composition is administered with an antibiotic. Examples of particular classes of antibiotics useful for synergistic therapy with the composition of the invention include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines and macrolides (e.g., erythromycin and clarithromycin). Further to the antibiotics listed above, typical antibiotics include aminoglycosides (amikacin, gentamicin, kanamycin, netilmicin, tobramycin, streptomycin, azithromycin, clarithromycin, erythromycin, erythromycin estolate/ethylsuccinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin). Other classes of antibiotics include carbapenems (e.g., imipenem), monobactams (e.g., aztreonam), quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, teicoplanin). Other antibiotics include chloramphenicol, clindamycin, trimethoprim, sulfa-methoxazole, nitrofurantoin, rifampin, mupirocin and the cationic peptides.

Any of the foregoing methods of the invention further include a step of assessing the efficacy of the therapeutic treatment. Because the factors of the invention have a demonstrable ability to induce anti-apoptosis, survival, and/or proliferation of a target cell, the efficacy of the therapeutic treatment can be assessed by measuring aspects of the respective biological pathways, including measuring levels of factors (e.g., in the serum) that are associated with such pathways.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Secretion of Liver Regeneration Factors

Objectives

The purpose of this study was to evaluate the ability of C3A cells present in an active cartridge of the system of the disclosure to secrete factors that are reported in the literature as having a beneficial effect on hepatocyte replication and/or liver regeneration.

Materials and Methods

C3A cell cartridge spent media were assayed using chemiluminescent multiplex array detection (Aushon) and/or contracted immunoassay multiplex services (Myriad Rules Based Medicine) for known mitogens, angiogenesis factors, or other proteins demonstrated in the literature to be involved with liver regeneration. System steady-state concentrations were converted into a “Dose” by multiplying the perfusion flow rates and time, then compared to literature values of normal healthy individuals, and a mass that a Dose may be expected to increase above those levels was determined.

Results

TABLE III
Secreted Factors

| | System | | | | Normal | | Normal | | System | |
|---|---|---|---|---|---|---|---|---|---|---|
| | Levels | | Dose¹ | | Serum | | Serum | | Additive | |
| Factor | (per cartridge) | | (4 cartridges) | | Levels | | Amount² | | Amount³ | |
| HGF | 50 | pg/mL | 1.5 | μg/d | 574 | pg/mL | 1.7 | μg | 0.5 | ng |
| TGFα | 400 | pg/mL | 11 | μg/d | 150 | pg/mL | 450 | ng | 3.6 | ng |
| Amphiregulin | 300 | pg/mL | 8.5 | μg/d | 200 | pg/mL | 60 | ng | 2.8 | ng |
| HB-EGF | 5.5 | pg/mL | 167 | ng/d | 5 | pg/mL | 15 | ng | 0.06 | ng |
| PDGF-BB | 190 | pg/mL | 5.5 | μg/d | 8.5 | ng/mL | 25.5 | ng | 1.8 | ng |
| VEGF | 117 | ng/mL | 3.5 | mg/d | 150 | pg/mL | 450 | ng | 1,167 | ng |
| VEGF-C | 140 | pg/mL | 4 | μg/d | 2.8 | ng/mL | 8.4 | pg | 1.3 | ng |
| PLGF | 850 | pg/mL | 25 | μg/d | 8 | pg/mL | 24 | ng | 8.3 | ng |
| ANG2 | 800 | pg/mL | 23 | μg/d | 1.1 | mg/mL | 3.3 | pg | 7.6 | ng |
| SCF | 75 | pg/mL | 2.1 | μg/d | 3.3 | ng/mL | 9.9 | pg | 0.7 | ng |
| EPO | 200 | mlU/ml | 5,760 | mlU/d | 4 27 | mU/mL | 12-81 | U | 1.9 | mlU |

¹Experimentally determined steady-state growth system concentrations × flow rate per cartridge × time.
²Normal serum levels × 3 L average body plasma volume.
³Dose × 3 L average body plasma volume.

Discussion

These data demonstrate that the C3A cells secrete a variety of proteins with reported involvement in liver regeneration, including growth, angiogenic and hematopoetic factors.

Most growth factors, cytokines, and hormones act through receptor tyrosine kinases on the cell surface to trigger intracellular signaling cascades. For hepatocytes, these include MET and EGFR. Ligands acting on Fas receptors, can signal apoptotic pathways. The integration of these multiple signaling pathways results in whether a cell is induced towards proliferation, survival, or apoptosis.

HGF is the most widely known hepatocyte mitogen, but other mitogens include transforming growth factor alpha (TGFα), amphiregulin, heparin-binding EGF (HB-EGF), and platelet-derived growth factor BB (PDGF-BB). All of these growth factors were secreted in measurable amounts by C3A cells.

In addition to hepatocyte mitogens, regenerating liver needs an increased vascular supply to support the increased tissue mass. Vascular endothelial growth factor (VEGF) is the most widely recognized angiogenic factor. It stimulates sinusoidal endothelial cells to secrete HGF. VEGF and other angiogenic factors, VEGF-C, placental growth factor (PLGF), and angiopoiein2 (ANG2) were produced by C3A cells.

Also secreted by C3A cells were stem cell factor (SCF) and erythropoietin (EPO). SCF stimulates hematopoiesis of myeloid cells, acts synergistically with GM-CSF to induce proliferation in cholangiocytes and hepatocytes, and increases in C3A cells in response to IL-1β exposure. EPO improves survival in rat hepatectomy models, with increased liver weights and higher mitotic indices.

These proteins could act directly on hepatocyte populations or act indirectly on hepatocytes by stimulating other resident cell populations, such as endothelial cells, stellate cells, or immune cells to produce hepatocyte-beneficial factors.

Further, it has been shown that many of these constitutively secreted factors are upregulated when C3A cells are exposed to pro-inflammatory cytokines, as may be present in patients with hepatitis. Thus levels of protein secretion may be increased further during patient treatment.

These data suggest C3A cells contribute to liver regeneration by providing an environment of pro-hepatocyte mediators as part of the multiple mechanisms for therapeutic benefit.

Conclusions

C3A cells are capable of producing a variety of secreted factors with known involvement in liver regeneration. This may facilitate liver regeneration, either directly through direct stimulation of hepatocytes, indirectly through interactions with other resident cell populations during treatment of liver failure patients with the system of the disclosure.

### Example 2

### Promoting Anti-Apoptosis, Survival and/or Proliferative Capacity of Liver Cells

The present study demonstrates a potential role for C3A cell secreted factors in a subsequent stage of liver regeneration, that of promoting cell survival and proliferative capacity of various liver cell types.

Objectives

The purpose of this study was to evaluate the ability of C3A cells to secrete factors reported in the literature as having a beneficial effect on hepatocyte survival, replication and/or liver regeneration. Then, finding such factors, to evaluate the effects of selected factors on various liver cell types.

Materials and Methods

The system of the disclosure is a human hepatic cell-based liver treatment comprised of four metabolically-active cell cartridges (C3A cells) with ancillary device components and support circuitry intended to continuously treat subjects with liver failure secondary to acute hepatocellular insult and alcohol use. C3A cell cartridge spent media were assayed using contracted ELISA multiplex (Myriad) or chemiluminescent multiplex array detection (Aushon Ciraplex) assays for known mitogenic, angiogenic and other regenerative factors.

A primary human hepatocyte (PHH) apoptosis model was adapted from Berasain et al. (J Biol Chem. 280(19):19012-20 (2005)). Apoptosis was induced in PHH (Gibco) using anti-CD95 (Fas) antibody (EOS9.1, eBioscience) following a 3-h incubation with Williams E medium (w/supplements, w/o dexamethasone, [Gibco]) or system conditioned media (CM) prepared by static incubation of Williams E medium in a mature C3A cell cartridge. Apoptosis was measured by Caspase-Glo 3/7 Assay (Promega), annexin V (Roche) and Western immunoblot (primary antibodies, Cell Signaling).

A human aortic endothelial cell (HAEC) angiogenic factor model was developed as a surrogate for liver sinusoidal EC (LSEC) by co-culture in Transwells with C3A cells or treated with CM prepared by static incubation of EGM-2 media (Lonza) in a mature cartridge. Cumulative expression of selected angiogenic factors was measured in supernatants at 24, 48 and 72 h by Aushon Ciraplex.

Results

C3A cell cartridge spent media.

Evaluation of system spent media (media collected from mature cartridges, maintained under flow, at steady-state conditions) showed that the C3A cells produce a number of recognized growth and angiogenic factors (Table III of Example 1).

To assess the potential effects of these factors on the various cells of the liver, a series of cell-based models were developed.

PHH Apoptosis Model.

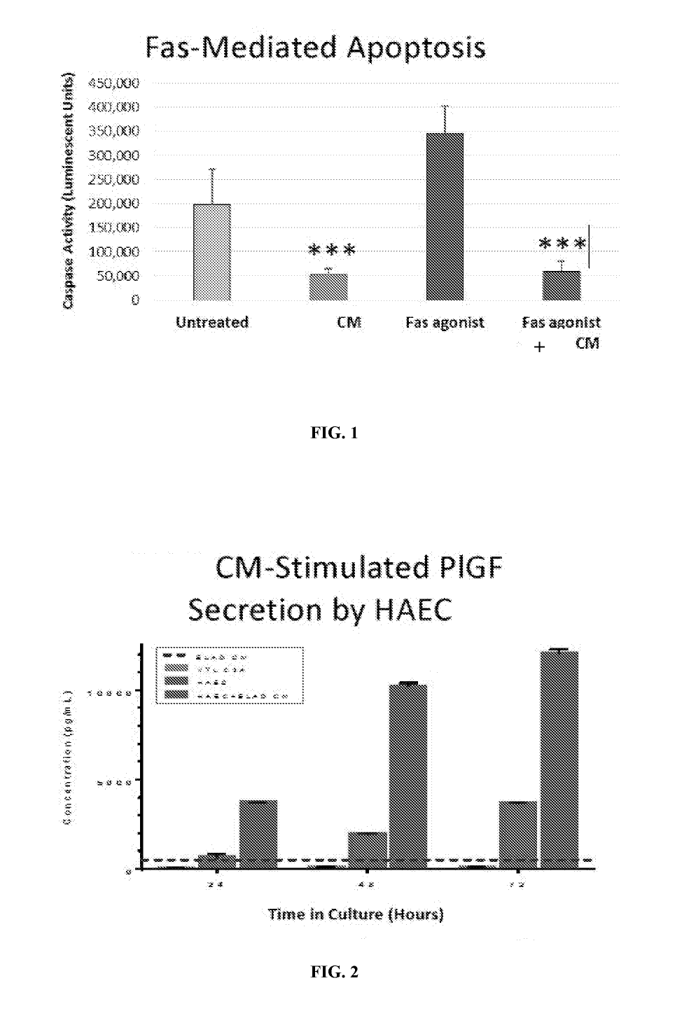

CM administered 3 h prior to challenge of PHH cultures with a Fas-agonist antibody significantly reduced Fas mediated apoptosis, as measured by caspase activity (FIG. 1). CM also reduced spontaneous apoptosis in untreated hepatocytes. CM-treated PHH maintained a more normal size and cobblestone morphology vs. Fas-agonist-treated PHH, as visualized by annexin V staining (data not shown).

Western immunoblots showed phosphorylation of signaling proteins associated with the EGFR (AKT, ERK1/2 and STAT3) in lysates from cells treated with CM or CM plus Fas-agonist. However, there was also phosphorylation of these same signaling proteins in the untreated control and to a varying extent the Fas-treated cells (data not shown).

HAEC Angiogenic Factor Model.

CM administered daily over 72 h significantly increased PLGF secretion by HAEC in a time-dependent manner (FIG. 2).

### FIGURE LEGENDS

FIG. 1: CM Reduces Apoptosis in PHH, Caspase activity was reduced in both untreated and Fas agonist-treated PHH cultures 3n the presence of CM. Error is SD of n=8 wells in 96-well format (***p<0.001 for all comparisons except two CM-treated vs each other. One-way ANOVA with Tukey post-hoc test).

FIG. 2: CM Increases PLGF Secretion by HAEC. HAEC cultures secrete significantly more PLGF in the presence of CM than EGM-2 media. Error bars are SD of n=2 replicates in 24-well format.

Discussion

Liver regeneration is a highly orchestrated event involving multiple pathways and cell types. Metabolically-active C3A cells offer the potential of contributing to liver regeneration by impacting these multiple cell types and pathways in ways that non-cell based therapies are unlikely to achieve.

This study highlights eleven factors secreted by C3A cells with recognized roles in cell growth, survival, regeneration, and hematopoiesis. The steady-state amount of each factor produced by four active cartridges during manufacturing is compared in Table III with normal serum values. Pharmacokinetic modeling of expected plasma concentrations in treated subjects is not offered here.

To begin to assess the potential effects of these factors on cells of the liver and to better understand the mechanisms of action of the system, CM was administered to PHH in culture. CM was found to promote survival in both untreated cells and those induced toward apoptosis by a Fas-agonist antibody (FIG. 1).

A similar model was shown to be dependent upon AR, the most necessary EGFR ligand for liver regeneration after partial hepatectomy. However, AR was not protective at 20 nM in the model (data not shown). To determine if the prosurvival effect of CM was mediated by EGFR activation, cell lysates were evaluated from untreated and Fas-agonist-treated C3A cells with and without CM, by Western immunoblot. Phosphorylation of AKT, ERK1/2, and STAT3 was found, suggesting activation of the EGFR. The C3A cells secrete a number of EGFR ligands (TGFα, AR, HB-EGF). The pro-PHH survival effect of CM is consistent with data showing CM from HepG2 cells (parental cell line to C3A cells) contains essential factors to support human fetal hepatocyte growth in culture.

LSEC and bone marrow progenitor cells of LSEC (BMSPC) have been shown to participate in liver regeneration by increased production of HGF in response to hepatic VEGF. The effects of C3A cell secreted VEGF was evaluated in an HAEC co-culture surrogate model of LSEC (due to greater availability of HAEC). Although HGF was not significantly increased (DNS), secretion of PLGF increased 5-fold over untreated HAEC, 24 h after administration of CM. The HAEC continued to produce increased PLGF in the presence of CM for the 72-h length of the model (FIG. 2). PLGF is purported to recruit VEGFR1+ stem cells from bone marrow for organogenesis.

Both SCF and EPO work synergistically with G-CSF; SCF to induce proliferation in cholangiocytes and hepatocytes, and EPO to increase survival in patients with decompensated cirrhosis. G-CSF secretion increases in C3A cells in response to IL-1β and IL-6 (data not shown).

Conclusions

C3A cells of the disclosure produce a variety of secreted factors with well-established roles in cell growth, survival, regeneration, and hematopoiesis. The cell-based models prevented PHH apoptosis and enhanced HAEC PLGF secretion. This may facilitate liver regeneration, directly by stimulation of hepatocytes, or indirectly by interactions with other resident cell populations during treatment.

### Example 3

### C3A Cells Inhibit Fas-Induced Apoptosis in Primary Human Hepatocytes Via Epidermal Growth Factor Receptor (EGFR) Activation and Secretion of Soluble Fas (sFAS)

Hallmarks of alcoholic hepatitis (AH) are increased hepatocellular death, increased liver dysfunction and further inflammatory responses if dying cells are ineffectively cleared. The inventors are clinically evaluating the system of the disclosure using C3A cells of the disclosure in the treatment of severe acute AH (sAAH). The inventors previously showed (Example 2) that conditioned medium (CM) from C3A cells grown in a three-dimensional bioreactor contains hepatocyte mitogens (amphiregulin, TGFα, HGF, HB-EGF, and PDGF-BB) and can inhibit Fas-induced apoptosis in primary human hepatocyte (PHH) cultures, as measured by caspase 3/7 activity and annexin V staining; however, the mechanism was previously unknown.

It was hypothesized that epidermal growth factor receptor (EGFR) activation by ligands in the CM may be responsible for the observed hepatoprotective effects. The purpose of this study was to determine the mechanism by which CM promotes hepatocyte survival in a model of Fas-induced apoptosis.

Apoptosis was induced in PHH in vitro by an anti-Fas agonist antibody. Addition of CM significantly inhibited apoptosis as measured by caspase-3/7 activity and annexin V staining, confirming previously reported results. New data using Western immunoblotting to detect caspase-8 cleavage products, as a measure of apoptosis, showed patterns consistent with activation of the EGFR by CM. Fas agonist-treated PHH lysates showed increased cleavage products, whereas lysates from PHH treated with Fas agonist in the presence of CM showed a reduction of cleavage products compared to controls. Further, addition of the EGFR-inhibitor canertinib to Fas agonist/CM-treated PHH produced cleavage product levels similar to Fas agonist alone.

Phosphorylation of proteins known to be associated with EGFR activation (e.g. MEK 1/2, ERK 1/2, and STAT3) were increased in lysates of CM-treated PHH and were decreased in samples treated with canertinib.

Treatment with recombinant human amphiregulin reduced PHH apoptosis, an effect blocked when canertinib was added to the treatment. However, the hepatoprotective effect of amphiregulin was less than that of CM, suggesting that an additional mechanism and/or EGFR ligand may be involved.

C3A cells were found to produce soluble Fas (sFas). Recombinant human sFas was effective in reducing apoptosis in PHH, supporting secretion of sFas by C3A cells as an additional and novel factor contributing to survival of PHH in this Fas-induced apoptosis model.

These results demonstrate that C3A cells promote hepatocyte survival through multiple mechanisms and suggest potential means by which treatment with the present system may provide benefit to sAAH subjects.

Although the invention has been described with reference to the above example, it will be understood that modifications and variations are encompassed within the spirit and scope of the invention. Accordingly, the invention is limited only by the following claims.

1. A composition for inducing anti-apoptosis, anti-pyroptosis, anti-necroptosis, protection, survival, and/or proliferation, and/or phenotypic modulation of a target cell, the composition comprising one or more anti-apoptotic, pro-survival, and/or pro-regeneration factors, wherein the one or more factors are selected from those set forth in Table I, II or III.

2. The composition of claim 1, wherein the factors are secreted from a source cell.

3. The composition of claim 2, wherein the source cell is a eukaryotic cell.

4. The composition of claim 1, wherein the source cell is a mammalian cell.

5. The composition of claim 4, wherein the source cell is a human cell.

6. The composition of claim 1, wherein the source cell is a hepatocyte.

7. The composition of claim 1, wherein the source cell is a recombinantly engineered cell.

8. The composition of claim 1, wherein the source cell is a hepatoblastoma-derived cell.

9. The composition of claim 1, wherein the source cell is a HepG2 cell or a C3A cell.

10. The composition of claim 9, wherein the source cell is a clonal derivative from a parental C3A cell line.

11. The composition of claim 1, wherein the target cell is a mammalian cell.

12. The composition of claim 11, wherein the target cell is a human cell.

13. The composition of claim 1, wherein the target cell is a liver-derived cell.

14. The composition of claim 1, wherein the target cell is a hepatoblastoma-derived cell.

15. The composition of claim 1, wherein the target cell is a cell of a diseased liver.

16. The composition of claim 15, wherein the disease is cirrhosis, hepatitis or fatty liver disease.

17. The composition of claim 1, wherein the factors comprise at least Amphiregulin (AR) or soluble Fas receptor.

18. The composition of claim 17, wherein the factors comprise at least Amphiregulin (AR) and soluble soluble Fas (sFas).

19. The composition of claim 18, wherein the factors further comprise one or more additional factors selected from these listed in Table 1.

20. The composition of claim 1, wherein the factors comprise one or more mitogens selected from those listed in Table 1.

21. The composition of claim 1, wherein the factors comprise one or more factors which inhibit apoptosis related signal transduction in non-disease related cells selected from those listed in Table 1.

22. The composition of claim 1, wherein the factors comprise one or more factors that promotes apoptosis in disease related cells selected from those listed in Table 1.

23. The composition of claim 1, wherein the factors comprise one or more factors that induces a phenotypic shift resulting in improved cellular functioning selected from those listed in Table 1.

24. The composition of claim 1, wherein the each of the plurality of factors is present at a concentration of at least 1, 10, 100, 1,000, 10,000, 100,000, 1,000,000 pg/ml or greater.

25. The composition of claim 1, wherein the factors induce apoptosis in activated stellate cells but not non-activated stellate cells.

26. The composition of claim 1, wherein the composition further comprises a eukaryotic cell.

27. The composition of claim 26, wherein the eukaryotic cell is a hepatocyte or hepatoblastoma-derived cell.

28. The composition of claim 26, wherein the eukaryotic cell is a recombinantly engineered cell.

29. The composition of claim 26, wherein the eukaryotic cell is a HepG2 cell, a C3A cell, or a clonal derivative from a parental C3A cell line.

30. A method of inducing anti-apoptosis, anti-pyroptosis, anti-necroptosis, protection, survival, and/or proliferation, and/or phenotypic modulation of a target cell comprising contacting the target cell with the composition according to claim 1, thereby inducing anti-apoptosis, anti-pyroptosis, anti-necroptosis, protection, survival, and/or proliferation, and/or phenotypic modulation of the target cell.

31-44. (canceled)

45. A method of treating a disease or disorder in a subject comprising administering a composition according to claim 1 to the subject, thereby treating the disease or disorder.

46-54. (canceled)

55. A qualified C3A cell line derived from a parental C3A cell line, wherein cells of the cell line express a plurality of factors as set forth in Table 1.
